(19) United States
(12) Patent Application Publication
(10) Pub. No.: US 2023/0349684 A1
(43) Pub. Date: Nov. 2, 2023

(54) SYSTEM AND METHOD FOR MEASURING ROCK VOLUME CHANGE UNDER MICROWAVE IRRADIATION

(71) Applicants: Shenzhen University, Shenzhen (CN); SICHUAN UNIVERSITY, Chengdu, Sichuan (CN)

(72) Inventors: Mingzhong Gao, Shenzhen (CN); Bengao Yang, Shenzhen (CN); Ruifeng Tang, Shenzhen (CN); Junjun Liu, Shenzhen (CN); Siqi Ye, Shenzhen (CN); Xuemin Zhou, Shenzhen (CN); Jun Wang, Shenzhen (CN); Haichun Hao, Shenzhen (CN); Zheng Gao, Shenzhen (CN); Yan Wu, Shenzhen (CN); Zhaoying Yang, Shenzhen (CN); Xiangyue Wen, Shenzhen (CN); Xuan Wang, Shenzhen (CN)

(21) Appl. No.: 17/919,448

(22) PCT Filed: Jul. 13, 2021
(86) PCT No.: PCT/CN2021/105974

§ 371 Date: Oct. 17, 2022

(30) Foreign Application Priority Data
Dec. 17, 2020 (CN) 202011496721.8

(51) Int. Cl. G01B 7/16; G01L 1/22; G01N 22/00; G01N 33/24

(52) U.S. Cl. CPC G01B 7/18; G01L 1/22; G01N 22/00; G01N 33/24

(57) ABSTRACT

A system and method for measuring rock volume change under microwave irradiation. The system includes a test cavity, a microwave control device, strain measurement devices, a measurement circuit, and a resistance strain gauge. The test cavity is of a sealed cavity structure, in which a rock specimen and the microwave control device are provided. The strain measurement device includes circumferential strain gauges and axial strain gauges, and is attached to the surface of the rock specimen. The measurement circuit is connected to the strain measurement devices by means of leads, and the resistance strain gauge. After microwaves emitted by the microwave control device act on the rock specimen, the plurality of circumferential strain gauges and the axial strain gauges collect data from the rock specimen, and the volume change of the rock specimen can be obtained in combination with the measurement circuit and the resistance strain gauge.

### TECHNICAL FIELD

The present disclosure relates to the technical field of rock mechanics equipment, relates to a system and method for measuring rock volume change, particularly to a system and method capable of accurately measuring the influence of microwave irradiation on rock volume.

### BACKGROUND

How to effectively crush rocks becomes a hot spot of research during mining of mineral resources deep under the earth's surface. The rocks are crushed mainly by using a drill bit in the traditional mode, and the original rock crushing mode is limited by factors such as overlarge rock strength and easy abrasion of the drill bit, resulting in the problems of low efficiency, high cost and the like.

In recent years, microwaves have been introduced into the field of rock crushing, the microwaves cause the overall temperature of an object subjected to the microwaves, to rise, by promoting mutual friction between molecules in the rocks to generate heat, so that the rock mass is softened to be conveniently crushed, which has the advantages of no secondary pollution and the like and has been proved to be technically and economically feasible in the field. Related researches have proved that the microwave irradiation can greatly reduce the strength of rocks and even cause rock to melt and peel, and the microwave technology has a good application prospect in the field of rock crushing.

Before microwaves are applied to the rock crushing field, the effect of microwaves on rock volume change needs to be verified in a laboratory. According to the existing rock volume change measuring mode, the rock volume change is indirectly measured through the volume change of liquid or gas, and in consideration of the microwave irradiation, the temperature of the rock suddenly rises, and the volume change of the liquid or gas is influenced. Therefore, the rock volume measurement result in the prior art is unreliable, and a novel rock volume change measurement solution needs to be proposed.

### SUMMARY

Aiming at the defects in the prior art, the present disclosure provides a system and method for measuring rock volume change under microwave irradiation, which can exclude the volume change of liquid or gas caused by temperature change, and accurately measure the influence of the microwave irradiation on the rock volume.

The technical solution adopted for solving the technical problem of the present disclosure is as follows.

A system for measuring rock volume change under microwave irradiation includes: a test cavity, a microwave control device, a strain measurement device, a measurement circuit and a resistance strain gauge;
where the test cavity is of a sealed cavity structure, and a rock specimen is placed in the test cavity;
the microwave control device is provided inside the test cavity and configured to provide microwaves acting on the rock specimen;
the strain measurement device includes a circumferential strain gauge and an axial strain gauge, where a plurality of circumferential strain gauges and a plurality of axial strain gauges are provided respectively, and the strain measurement device is stuck to a surface of the rock specimen; and
the measurement circuit is connected to the strain measurement device by means of a lead, and the resistance strain gauge is connected to the measurement circuit.

Compared with the prior art, the technical effects of the technical solution are as follows: after microwaves emitted by the microwave control device act on the rock specimen, data collection is performed on the rock specimen by means of the plurality of circumferential strain gauges and the axial strain gauges which are stuck to the surface of the rock specimen, and the volume change of the rock specimen can be obtained in combination with the measurement circuit and the resistance strain gauge. Therefore, the volume change of liquid or gas caused by temperature change is excluded, thereby accurately measuring the influence of the microwave irradiation on the rock volume.

Further, the strain measurement device includes sensitive grids, substrates and covering layers;
the sensitive grids, the substrates and the covering layers form laminated structures, the sensitive grids are located between the substrates and the covering layers, and the sensitive grids are connected to the measurement circuit by means of a lead; and
when the strain measurement device is stuck to the surface of the rock specimen, the substrates are located at positions close to the rock specimen, and the covering layers are located at positions away from the rock specimen.

Through the adoption of the solution above, the beneficial effects are: the sensitive grids, the substrates and the covering layers form the strain measurement device, and the sensitive grids are located between the substrates and the covering layers, so that the sensitive grids can be protected by the substrates and the covering layers, and furthermore, the measurement accuracy is improved.

Further, the covering layers are made of foamed silicon dioxide layer.

Through the adoption of the solution above, the beneficial effects are: the foamed silicon dioxide layer adopted as the covering layer can have a function of the corrosion-proof and moisture-proof effects, and prevent the sensitive grids from being subjected to microwave irradiation.

Further, in each circumferential strain gauge, the sensitive grids are arranged along a horizontal direction; and in each axial strain gauge, the sensitive grids are arranged along a vertical direction.

Through the adoption of the solution, the beneficial effects are: circumferential deformation data and axial deformation data of the rock specimen are collected, and deformation data of the rock specimen are collected in multiple directions.

Further, three to ten circumferential strain gauges are provided, and the sensitive grids on all circumferential strain gauges are parallel to one another; and
three to ten axial strain gauges are provided, and the sensitive grids on all axial strain gauges are parallel to one another.

Through the adoption of the solution above, the beneficial effects are: according to a size of the rock specimen, the numbers of the circumferential strain gauges and the axial strain gauges are adaptively adjusted, so as to improve the measurement accuracy.

Further, the microwave control device includes a microwave source, a waveguide assembly and a microwave transmitting disc, an end of the microwave source is connected to the waveguide assembly, and another end of the microwave source is connected to the microwave transmitting disc;
the test cavity includes a bottom plate, a side plate and a top plate, a rock bearing base platform is arranged on the bottom plate of the test cavity, and the rock specimen is placed on the rock bearing base platform; and
the microwave source is arranged on the top plate or the side plate of the test cavity, and the microwave transmitting disc transmits microwaves towards the rock specimen.

Through the adoption of the solution above, the beneficial effects are: the rock bearing base platform is arranged on the bottom plate of the test cavity, the microwave source is arranged on the top plate or the side plate of the test cavity, and the relative position relation between the microwave source and the rock specimen can be adjusted according to actual conditions, so as to improve the universality of the system.

Further, the microwave source includes a power supply assembly, a transformer assembly and a control circuit.

Through the adoption of the solution above, the beneficial effects are: the microwave source is formed by the power supply assembly, the transformer assembly and the control circuit, so that microwaves for testing are provided.

Further, the bottom plate, the side plate and the top plate are each of a plate-shaped double-layer structure composed of a metal layer cavity and a heat insulation layer cavity;
a taking and placing through hole is formed in the side plate, and a side opening door is arranged in the taking and placing through hole; and
the rock bearing base platform is arranged on the bottom plate through a height adjusting assembly.

Through the adoption of the solution above, the beneficial effects are: the metal layer cavity and the heat insulation layer cavity form the bottom plate, the side plate and the top plate, namely the whole test cavity is of a double-layer structure, so that better heat insulation and radiation prevention effects can be achieved, and the safety of the test process is ensured.

Further, the measurement circuit includes an amplifier and a bridge; and
an end of the lead is connected to the strain measurement device, another end of the lead is connected to the bridge through the amplifier, and the bridge is connected to the resistance strain gauge.

Through the adoption of the solution above, the beneficial effects are: electric signals collected by the strain measurement device are processed through the amplifier and the bridge, and the reliability of test data is improved.

According to a method for measuring rock volume change under microwave irradiation, the method for measuring rock volume change being based on the system for measuring rock volume change under microwave irradiation, the method includes following steps:
transmitting microwaves to the rock specimen through the microwave control device;
collecting a first resistance variation through the circumferential strain gauges and the measurement circuit, collecting a second resistance variation through the axial strain gauges and the measurement circuit after the microwaves are applied on the rock specimen, and inputting the collected first resistance variation and second resistance variation into the resistance strain gauge; and
obtaining rock circumferential volume strain according to the first resistance variation, obtaining rock axial volume strain according to the second resistance variation, and obtaining total volume application amount according to the rock circumferential volume strain and the rock axial volume strain by the resistance strain gauge.

Compared with the prior art, the technical effects of the technical solution are as follows: after microwaves emitted by the microwave control device act on the rock specimen, data collection is performed on the rock specimen by means of the plurality of circumferential strain gauges and the axial strain gauges which are stuck to the surface of the rock specimen, and the volume change of the rock specimen can be obtained, in combination with the measurement circuit and the resistance strain gauge. Therefore, the volume change of liquid or gas caused by temperature change is excluded, thereby accurately measuring the influence of the microwave irradiation on the rock volume.

### BRIEF DESCRIPTION OF THE DRAWINGS

FIG. 1 is a schematic diagram of a system for measuring rock volume change under microwave irradiation according to the present disclosure.

FIG. 2 is a schematic diagram of a microwave control device in the system for measuring rock volume change under microwave irradiation according to the present disclosure.

FIG. 3 is a schematic diagram of a strain measurement device in the system for measuring rock volume change under microwave irradiation according to the present disclosure.

FIG. 4 is a schematic diagram of a measurement circuit in the system for measuring rock volume change under microwave irradiation according to the present disclosure.

FIG. 5 is a flow diagram of a method for measuring rock volume change under microwave irradiation according to the present disclosure.

### REFERENCE NUMERALS

1 test cavity; 2 microwave control device; 3 strain measurement device; 4 measurement circuit; 5 lead; 6 resistance strain gauge;

101 rock bearing base platform; 102 height adjusting assembly;

201 microwave source; 202 waveguide assembly; 203 microwave transmitting disc;

301 circumferential strain gauge; 302 axial strain gauge; 303 sensitive grid; 304 substrate;

401 amplifier; and 402 bridge.

### DETAILED DESCRIPTION OF THE EMBODIMENTS

In order to make the objectives, technical solutions and advantages of the present disclosure clearer, the present disclosure will be further described hereinbelow with reference to the accompanying drawing and embodiments thereof. It shall be understood that, the embodiments described herein are only intended to illustrate but not to limit the present disclosure.

In the description of the present disclosure, it needs to be illustrated that the direction or position relations indicated by the terms such as “center”, “upper”, “lower”, “front”, “rear”, “left” and “right” are direction or position relations illustrated based on the accompanying drawings, just for facilitating the description of the present disclosure and simplifying the description, but not for indicating or hinting that the indicated device or element must be in a specific direction and is constructed and operated in the specific direction, the terms cannot be understood as the restriction on the present disclosure. Moreover, the terms such as “first” and “second” are just used for distinguishing the description, but cannot be understood to indicate or hint relative importance.

In the description of the present disclosure, it needs to be illustrated that, except as otherwise noted, the terms such as “install”, “link” and “connect” should be generally understood, for example, they can means that, components can be fixedly connected, and also can be detachably connected or integrally connected; the components can be mechanically connected, and also can be electrically connected; the components can be directly connected and also can be indirectly connected through an intermediate, and they also can means internal communication between two components. When one element is referred to as being “fixed” or “arranged” on the other element, it can be directly on the other element or indirectly on the other element. When one element is referred to as being “connected” to the other element, it may be directly connected to the other element or indirectly connected to the other element. For those skilled in the art, the specific meanings of the terms in the present disclosure can be understood according to specific conditions.

Before microwave is applied to the rock crushing field on a large scale, the effect of microwave on rock volume change needs to be verified in a laboratory. According to the existing rock volume change measuring mode, the rock volume change is indirectly measured through the volume change of liquid or gas, and in consideration of the microwave irradiation, the temperature of the rock suddenly rises, and the volume change of the liquid or gas is influenced. Therefore, the rock volume measurement result in the prior art is unreliable. Briefly, in the prior art, when the rock volume change is studied, the volume change of liquid or gas around the rock caused by the rock volume change is used for indirectly measuring the rock volume change. For example, the rock specimen is placed in a container filled with liquid, a part of the liquid around the rock specimen is discharged when the volume of the rock is increased. In the prior art, the rock volume change is indirectly measured by measuring the volume of the discharged liquid. However, when the microwaves act on the rock specimen, the microwaves influence the volume of the liquid, and the volume of the discharged liquid is difficult to accurately reflect the rock volume change, which is the disadvantage of the prior art. Therefore, a novel rock volume change measurement solution needs to be proposed.

As shown in FIG. 1, a system for measuring rock volume change under microwave irradiation includes a test cavity 1, a microwave control device 2, a strain measurement device 3, a measurement circuit 4, and a resistance strain gauge 6.

The test cavity 1 is of a sealed cavity structure, and a rock specimen is placed in the test cavity 1. The microwave control device 2 is provided inside the test cavity 1 and configured to provide microwaves on the rock specimen. The strain measurement device 3 includes circumferential strain gauges 301 and axial strain gauges 302, a plurality of circumferential strain gauges 301 and a plurality of axial strain gauges 302 are provided respectively, and the strain measurement device 3 is stuck to the surface of the rock specimen. The measurement circuit 4 is connected to the strain measurement device 3 by means of a lead 5, and the resistance strain gauge 6 is connected to the measurement circuit 4.

A core of the present disclosure lies in that the plurality of circumferential strain gauges 301 and the plurality of axial strain gauges 302 used as the strain measurement device 3 are configured for directly measuring the volume change of the rock specimen. The plurality of circumferential strain gauges 301 are arranged on the rock specimen to collect a plurality of circumferential deformation data on the rock specimen. Furthermore, the plurality of axial strain gauges 302 are arranged on the rock specimen to collect a plurality of axial deformation data on the rock specimen. Through the plurality of circumferential deformation data and the plurality of axial deformation data, the overall deformation condition of the rock specimen can be accurately reflected.

Therefore, based on the technical solution above, after microwaves emitted by the microwave control device 2 act on the rock specimen, data collection is performed on the rock specimen by means of the plurality of circumferential strain gauges 301 and the axial strain gauges 302 which are stuck to the surface of the rock specimen, and the volume change of the rock specimen can be obtained, in combination with the measurement circuit 3 and the resistance strain gauge 6. Therefore, the volume change of liquid or gas caused by temperature change is excluded, and the influence of the microwave irradiation on the rock volume is measured more accurately.

In some embodiments, the strain measurement device 3 includes sensitive grids 303, substrates 304 and covering layers; the sensitive grids 303, the substrates 304 and the covering layers form laminated structures, the sensitive grids 303 are located between the substrates 304 and the covering layers, and the sensitive grids 303 are connected to the measurement circuit 4 by means of the lead 5. When the strain measurement device 3 is stuck to the surface of the rock specimen, the substrates 304 are located at positions close to the rock specimen, and the covering layers are located at positions away from the rock specimen. The sensitive grids 303, the substrates 304 and the covering layers form the strain measurement device 3, and the sensitive grids 303 are located between the substrates 304 and the covering layers, so that the sensitive grids 303 can be protected by the substrates 304 and the covering layers, and furthermore, the measurement accuracy is improved.

In the test process, the sensitive grids 303 may be damped or even corroded, resulting in failure of the strain measurement device 3. In order to avoid the problem, in some embodiments, the covering layer is made of foamed silicon dioxide layer. The foamed silicon dioxide layer adopted as the covering layer can have a function of corrosion-proof and moisture-proof effects and prevent the sensitive grids 303 from being subjected to microwave irradiation. Similarly, a foamed silicon dioxide sleeve is arranged outside the lead 5, and the lead 5 is prevented from being damped and corroded through the foamed silicon dioxide sleeve.

Specifically, the substrate 304 is an insulating substrate 304. The sensitive grids 303 are pasted on the substrates 304, and the sensitive grids 303 are configured for converting the strain of the rock specimen into the resistance variation. Moreover, in the process, the substrates 304 are arranged between the sensitive grids 303 and the rock specimen and can play an insulating role. In addition, the covering layer is made of an organic polymer material, so that the sensitive grids 303 are protected from radiation damage and mechanical damage, and the covering layer has good mechanical characteristics. The sensitive grids 303 and the measurement circuit 4 are communicated by the lead 5, particularly using double leads 5 welded in multiple points.

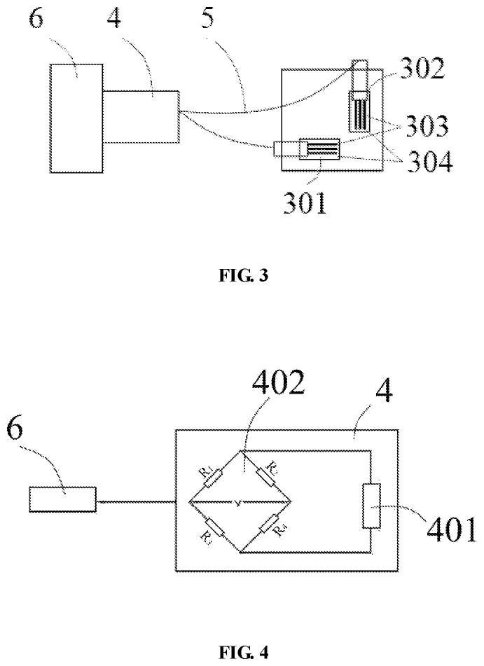

As shown in FIG. 3, in some embodiments, in each circumferential strain gauge 301, the sensitive grids 303 are arranged along the horizontal direction; and in each axial strain gauge 302, the sensitive grids 303 are arranged along the vertical direction. Circumferential deformation data of the rock specimen are collected through the horizontally arranged sensitive grids 303, axial deformation data are collected through the vertically arranged sensitive grids 303, and deformation data of the rock specimen are collected in multiple directions. Specifically, three to ten circumferential strain gauges 301 are provided, and the sensitive grids 303 on all circumferential strain gauges 301 are parallel to one another; and three to ten axial strain gauges 302 are provided, and the sensitive grids 303 on all axial strain gauges 302 are parallel to one another. According to the size of the rock specimen, the numbers of the circumferential strain gauges 301 and the axial strain gauges 302 are adaptively adjusted to improve the measurement accuracy.

For example, when a size of the rock specimen is 50 mm in diameter and 100 mm in height, five circumferential strain gauges 301 and five axial strain gauges 302 are provided. Considering that mineral components and internal structure of the rock specimen are complex and diversified, and expanded volumes of mineral components and internal structure when heated are possibly inconsistent, in order to keep the accuracy of volume change measurement as far as possible and consider actual needs, in the test process, the rock specimen with the diameter of 50 mm and the height of 100 mm are divided into five sections for measuring the strain of each section, and a strain gauge is arranged at a middle point of each of the five sections to measure the circumferential and axial strains of the section, and the deformation of each section is approximate to uniform deformation, so as to obtain that the overall volume variation.

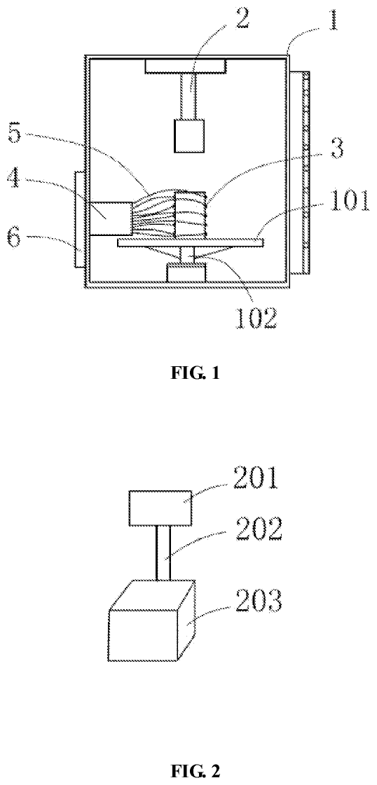

As shown in FIG. 1 and FIG. 2, the microwave control device 2 includes a microwave source 201, a waveguide assembly 202 and a microwave transmitting disc 203, one end of the microwave source 201 is connected to the waveguide assembly 202, and the other end of the microwave source 201 is connected to the microwave transmitting disc 203. The test cavity 1 includes a bottom plate, a side plate and a top plate, a rock bearing base platform 101 is arranged on the bottom plate of the test cavity 1, and the rock specimen is placed on the rock bearing base platform 101; and the microwave source 201 is arranged on the top plate or the side plate of the test cavity 1, and the microwave transmitting disc 203 transmits microwaves towards the rock specimen. The rock bearing base platform 101 is arranged on the bottom plate of the test cavity 1, the microwave source 201 is arranged on the top plate or the side plate of the test cavity 1, and the relative position relation between the microwave source 201 and the rock specimen can be adjusted according to actual conditions, so that the universality of the system is improved. Specifically, the microwave source 201 includes a power supply assembly, a transformer assembly and a control circuit. The microwave source 201 is formed by the power supply assembly, the transformer assembly and the control circuit, to provide microwaves for testing.

The microwave control device 2 including the microwave source 201, the waveguide assembly 202 and the microwave transmitting disc 203 is configured to convert electric energy into microwave energy, so that the effect of quick rock breaking is achieved by transmitting microwaves to heat rock. The circumferential strain gauges 301 and the axial strain gauges 302 are pasted on the rock specimen, and each strain gauge is uniformly distributed at the middle point of each section of the rock specimen, and the strain gauges can measure the circumferential strain and the axial strain at the same time.

The bottom plate, the side plate and the top plate each are of a plate-shaped double-layer structure composed of a metal layer cavity and a heat insulation layer cavity. A taking and placing through hole is formed in the side plate, and a side opening door is arranged in the taking and placing through hole. The rock bearing base platform 101 is arranged on the bottom plate through a height adjusting assembly 102. The metal layer cavity and the heat insulation layer cavity form the bottom plate, the side plate and the top plate, namely the whole test cavity 1 is of a double-layer structure, so that better heat insulation and radiation prevention effects can be achieved, and the safety of the test process is ensured. Moreover, the height adjusting assembly 102 may adjust the height of the rock bearing base platform 101 according to the size of the rock specimen, so as to adjust the relative position between the rock specimen and the microwave control device 2.

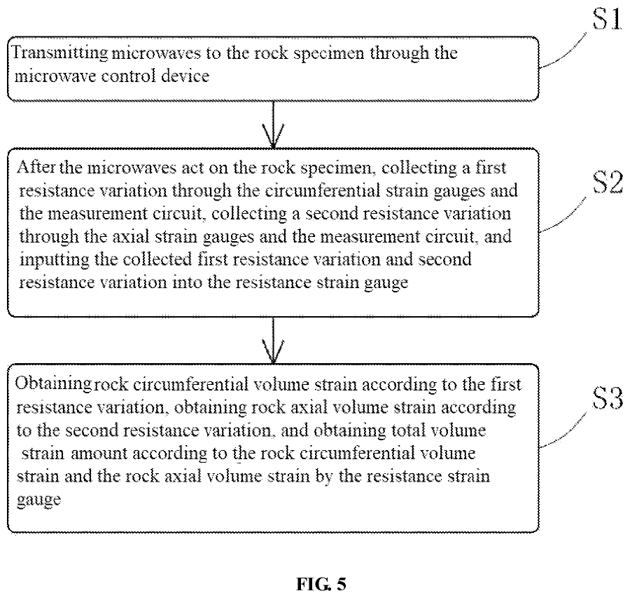

As shown in FIG. 4, the measurement circuit 4 includes an amplifier 401 and a bridge 402; one end of the lead 5 is connected to the strain measurement device 3, the other end of the lead 5 is connected to the bridge 402 through the amplifier 401, and the bridge 402 is connected to the resistance strain gauge 6. Electric signals collected by the strain measurement device 3 are processed through the amplifier 401 and the bridge 402, so as to improve the reliability of test data.

As shown in FIG. 5, according to a method for measuring rock volume change under microwave irradiation, the method for measuring rock volume change being based on the system for measuring rock volume change under microwave irradiation, the method includes the following steps:
S1, transmitting microwaves to the rock specimen through the microwave control device 2;
S2, collecting a first resistance variation through the circumferential strain gauges 301 and the measurement circuit 4 after the microwaves are applied on the rock specimen, collecting a second resistance variation through the axial strain gauges 302 and the measurement circuit 4, and inputting the collected first resistance variation and second resistance variation into the resistance strain gauge 6; and
S3, obtaining rock circumferential volume strain according to the first resistance variation, obtaining rock axial volume strain according to the second resistance variation, and obtaining total volume strain amount according to the rock circumferential volume strain and the rock axial volume strain by the resistance strain gauge 6.

It should be noted that the measurement circuit 4 and the resistance strain gauge 6 are configured for automatically converting the resistance variation into the volume variation, which is the conventional application of the measurement circuit 4 and the resistance strain gauge 6 in the prior art. Taking arrangement of five circumferential strain gauges 301 and five axial strain gauges 302 as an example, the specific process of analyzing the rock volume variation is illustrated.

An original height of the rock specimen is set to be l, a radius of a cylinder in a cross section is set to be r, a cylinder infinitesimal element is taken from the rock specimen, the side lengths of all the sides are dl and dr, and the volume dV of the infinitesimal element before deformation is set to be dV.

dV=πd/·(dr)²

After deformation, the axial strain measured by the strain gauge at each section is εᵢ, and the circumferential strain measured by the strain gauge at each section is εᵢ′ (i=1, 2, 3, 4 and 5).

Deformation at each segment can be approximate to uniform deformation, and the length changes of the sides after deformation are respectively as follows:

(1+εᵢ)dl, (1+εᵢ)dr.

The volume of the deformed infinitesimal element is dV=π(1+εᵢ)dl·(1+εᵢ′)²(dr)², and then the volume strain θ is:

$$θ = \frac{π ( 1 + {ε}_{i} ) dl \cdot {( 1 + {ε}_{i}^{′} )}^{2} dr - π dl \cdot {( dr )}^{2}}{π dl \cdot {( dr )}^{2}} .$$

The above calculation is simplified and higher-order trace is ignored to obtain the following formula:

θᵢ=εᵢ+2εᵢ′

The original volume of each section is set to be Vᵢ⁰, and the volume of each section after deformation is set to be Vi. And then, the total volume of the rock specimen is as follows:

$$V = {\sum }_{i = 1}^{5} ( 1 + {θ}_{i} ) {V}_{i}^{0} ,$$

where, (l=1, 2, 3, 4, 5).

In conclusion, the present disclosure provides the system and method for measuring rock volume change under microwave irradiation. The system includes the test cavity 1, the microwave control device 2, the strain measurement device 3, the measurement circuit 4, and the resistance strain gauge 6. The test cavity 1 is of a sealed cavity structure, and a rock specimen is placed in the test cavity 1. The microwave control device 2 is provided inside the test cavity 1 and configured for providing microwaves on the rock specimen. The strain measurement device 3 includes a circumferential strain gauge 301 and an axial strain gauge 302, a plurality of circumferential strain gauges 301 and a plurality of axial strain gauges 302 are provided respectively, and the strain measurement device 3 is stuck to the surface of the rock specimen. The measurement circuit 4 is connected to the strain measurement device 3 by means of the lead 5, and the resistance strain gauge 6 is connected to the measurement circuit 4. In consideration of uneven deformation generated after the rock specimen is heated by microwaves, circumferential deformation and axial deformation of each section of the rock specimen are measured by dividing the rock specimen into multiple sections, deformation of each section of the rock specimen is approximate to uniform deformation, and therefore the overall volume of the rock specimen after deformation is obtained, volume change of liquid or gas caused by temperature change is excluded, thereby accurately measuring the influence of the microwave irradiation on the rock volume.

It should be understood that the application of the present disclosure is not limited to the examples described above, and these modifications or variations can be made according to the above description for those skilled in the art, all of which are intended to fall within the scope of the appended claims.

1. A system for measuring rock volume change under microwave irradiation, comprising a test cavity, a microwave control device, a strain measurement device, a measurement circuit and a resistance strain gauge,
wherein the test cavity is of a sealed cavity structure, and a rock specimen is placed in the test cavity;
the microwave control device is provided inside the test cavity and configured to provide microwaves acting on the rock specimen;
the strain measurement device comprises a circumferential strain gauge and an axial strain gauge, wherein a plurality of circumferential strain gauges and a plurality of axial strain gauges are provided respectively, and the strain measurement device is stuck to a surface of the rock specimen; and
the measurement circuit is connected to the strain measurement device by means of a lead, and the resistance strain gauge is connected to the measurement circuit.

2. The system for measuring rock volume change under microwave irradiation according to claim 1, wherein the strain measurement device comprises sensitive grids, substrates and covering layers;
the sensitive grids, the substrates and the covering layers form laminated structures, the sensitive grids are located between the substrates and the covering layers, and the sensitive grids are connected to the measurement circuit by means of the lead; and
when the strain measurement device is stuck to the surface of the rock specimen, the substrates are located at positions close to the rock specimen, and the covering layers are located at positions away from the rock specimen.

3. The system for measuring rock volume change under microwave irradiation according to claim 2, wherein the covering layers are made of foamed silicon dioxide layer.

4. The system for measuring rock volume change under microwave irradiation according to claim 2, wherein in each circumferential strain gauge, the sensitive grids are arranged along a horizontal direction; and in each axial strain gauge, the sensitive grids are arranged along a vertical direction.

5. The system for measuring rock volume change under microwave irradiation according to claim 4, wherein three to ten circumferential strain gauges are provided, and the sensitive grids on all circumferential strain gauges are parallel to one another; and
three to ten axial strain gauges are provided, and the sensitive grids on all axial strain gauges are parallel to one another.

6. The system for measuring rock volume change under microwave irradiation according to claim 1, wherein the microwave control device comprises a microwave source, a waveguide assembly and a microwave transmitting disc, an end of the microwave source is connected to the waveguide assembly, and another end of the microwave source is connected to the microwave transmitting disc;
the test cavity comprises a bottom plate, a side plate and a top plate, a rock bearing base platform is arranged on the bottom plate of the test cavity, and the rock specimen is placed on the rock bearing base platform; and
the microwave source is arranged on the top plate or the side plate of the test cavity, and the microwave transmitting disc transmits microwaves towards the rock specimen.

7. The system for measuring rock volume change under microwave irradiation according to claim 6, wherein the microwave source comprises a power supply assembly, a transformer assembly and a control circuit.

8. The system for measuring rock volume change under microwave irradiation according to claim 6, wherein the bottom plate, the side plate and the top plate each are of a plate-shaped double-layer structure composed of a metal layer cavity and a heat insulation layer cavity;
a taking and placing through hole is formed in the side plate, and a side opening door is arranged in the taking and placing through hole; and
the rock bearing base platform is arranged on the bottom plate through a height adjusting assembly.

9. The system for measuring rock volume change under microwave irradiation according to claim 1, wherein the measurement circuit comprises an amplifier and a bridge; and
an end of the lead is connected to the strain measurement device, another end of the lead is connected to the bridge through the amplifier, and the bridge is connected to the resistance strain gauge.

10. A method for measuring rock volume change under microwave irradiation, the method for measuring rock volume change being based on a system for measuring rock volume change under microwave irradiation, wherein the system comprises a test cavity, a microwave control device, a strain measurement device, a measurement circuit and a resistance strain gauge,
wherein the test cavity is of a sealed cavity structure, and a rock specimen is placed in the test cavity;
the microwave control device is provided inside the test cavity and configured to provide microwaves acting on the rock specimen;
the strain measurement device comprises a circumferential strain gauge and an axial strain gauge, wherein a plurality of circumferential strain gauges and a plurality of axial strain gauges are provided respectively, and the strain measurement device is stuck to a surface of the rock specimen; and
the measurement circuit is connected to the strain measurement device by means of a lead, and the resistance strain gauge is connected to the measurement circuit;
the method comprises following steps:
transmitting microwaves to the rock specimen through the microwave control device;
collecting a first resistance variation through the circumferential strain gauges and the measurement circuit, collecting a second resistance variation through the axial strain gauges and the measurement circuit after the microwaves are applied on the rock specimen, and inputting the first resistance variation and the second resistance variation collected into the resistance strain gauge; and
obtaining rock circumferential volume strain according to the first resistance variation, obtaining rock axial volume strain according to the second resistance variation, and obtaining total volume strain amount according to the rock circumferential volume strain and the rock axial volume strain by the resistance strain gauge.

11. The method for measuring rock volume change under microwave irradiation according to claim 10, wherein the strain measurement device comprises sensitive grids, substrates and covering layers;
the sensitive grids, the substrates and the covering layers form laminated structures, the sensitive grids are located between the substrates and the covering layers, and the sensitive grids are connected to the measurement circuit by means of the lead; and
when the strain measurement device is stuck to the surface of the rock specimen, the substrates are located at positions close to the rock specimen, and the covering layers are located at positions away from the rock specimen.

12. The method for measuring rock volume change under microwave irradiation according to claim 11, wherein the covering layers are made of foamed silicon dioxide layer.

13. The method for measuring rock volume change under microwave irradiation according to claim 11, wherein in each circumferential strain gauge, the sensitive grids are arranged along a horizontal direction; and in each axial strain gauge, the sensitive grids are arranged along a vertical direction.

14. The method for measuring rock volume change under microwave irradiation according to claim 13, wherein three to ten circumferential strain gauges are provided, and the sensitive grids on all circumferential strain gauges are parallel to one another; and
three to ten axial strain gauges are provided, and the sensitive grids on all axial strain gauges are parallel to one another.

15. The method for measuring rock volume change under microwave irradiation according to claim 10, wherein the microwave control device comprises a microwave source, a waveguide assembly and a microwave transmitting disc, an end of the microwave source is connected to the waveguide assembly, and another end of the microwave source is connected to the microwave transmitting disc;
the test cavity comprises a bottom plate, a side plate and a top plate, a rock bearing base platform is arranged on the bottom plate of the test cavity, and the rock specimen is placed on the rock bearing base platform; and
the microwave source is arranged on the top plate or the side plate of the test cavity, and the microwave transmitting disc transmits microwaves towards the rock specimen.

16. The method for measuring rock volume change under microwave irradiation according to claim 15, wherein the microwave source comprises a power supply assembly, a transformer assembly and a control circuit.

17. The method for measuring rock volume change under microwave irradiation according to claim 15, wherein the bottom plate, the side plate and the top plate each are of a plate-shaped double-layer structure composed of a metal layer cavity and a heat insulation layer cavity;
a taking and placing through hole is formed in the side plate, and a side opening door is arranged in the taking and placing through hole; and
the rock bearing base platform is arranged on the bottom plate through a height adjusting assembly.

18. The method for measuring rock volume change under microwave irradiation according to claim 10, wherein the measurement circuit comprises an amplifier and a bridge; and
an end of the lead is connected to the strain measurement device, another end of the lead is connected to the bridge through the amplifier, and the bridge is connected to the resistance strain gauge.
